(19) United States
(12) Patent Application Publication
(10) Pub. No.: US 2016/0000378 A1
(43) Pub. Date: Jan. 7, 2016

(54) Human Health Property Monitoring System

(71) Applicant: David R. Hall, Provo, UT (US)

(72) Inventors: David R. Hall, Provo, UT (US); Aden K. Andrus, Spanish Fork, UT (US); B. Keoki Andrus, Spanish Fork, UT (US); Joshua Larsen, Spanish Fork, UT (US); Jared Reynolds, Provo, UT (US); Stephen Davis, Salt Lake City, UT (US); Huijae Kim, Provo, UT (US); Austen Schulz, Salt Lake City, UT (US)

(21) Appl. No.: 14/702,723

(22) Filed: May 2, 2015

Related U.S. Application Data
(60) Provisional application No. 61/987,838, filed on May 2, 2014.

(51) Int. Cl. A61B 5/00; G06F 17/18; A61B 10/00; A61B 5/1455; A61B 5/0205; A61B 5/145

(52) U.S. Cl. CPC A61B 5/7271; A61B 5/02055; A61B 5/14532; A61B 10/007; A61B 10/0038; A61B 5/0075; A61B 5/1455; G06F 17/18

(57) ABSTRACT

The present invention describes a system and method for assessing, monitoring and predicting disease and/or disease progression through ongoing and longitudinal analysis of various health-related parameters.

### FIELD OF THE INVENTION

The present invention relates to systems and methods for the in vitro detection and evaluation of analytes in urine and/or feces having one or more analytical tools incorporated into a toilet stool. Data collected may be processed by an integrated or remote processor to provide information about one or more analytes.

### BACKGROUND

Urine is the ultimate byproduct of physiology in the human body and thus represents the cumulative result of various metabolic processes. As a result, human health can be accurately assessed by changes in urinary constituents. The analytical value of urine has been recognized and successfully employed for centuries; however, despite the ubiquitous excretion of this information-rich fluid, psychosocial attitudes towards urine have severely limited its health informing potential. Although urine is a continuous and compulsory source of information on an individual's health, urine is only tested intermittently, and an immense source of readily available health data is lost. Other biological tests such as blood testing are even more invasive and also tested infrequently, leaving individuals and their healthcare providers with mere snapshots of the underlying physiologic activities driving changes in their health. In an effort to compensate for the lack of longitudinal trend data, healthcare providers compare test results with population statistics. This provides some insight into population-relative health at a single point in time, but little true insight into the dynamic and unique health processes of the individual. However, by seamlessly integrating the urine analysis process into the obligatory urine collection process facilitated by the modern toilet, it would be possible to generate unprecedented longitudinal, user-specific, dynamic health information to individuals and healthcare providers. The proposed invention provides such a means for enabling the ongoing analysis necessary to support this novel approach to evaluating, monitoring, and predicting health.

Since handling urine is a distasteful activity few individuals are willing to perform on a regular basis, obtaining daily or multi-daily urine test results necessitates an automated sample acquisition and analysis process that occurs at the site of normal urine excretion: the toilet. Toilets designed to reduce the user interface have been suggested previously. Several patents have been issued for toilets that use reagent-impregnated test strips to qualitatively evaluate aspects of the individual's urine such as leukocyte esterase, nitrites, urobilinogen, protein, pH, hemoglobin, specific gravity, ketones, bilirubin or glucose via a color change produced by a chemical reaction with the component of interest. For example, U.S. Pat. No. 4,961,431 uses a valve to dispense urine onto a preloaded test strip. The strip is then held in front of a “urine analyzing device” and the analytical results are displayed digitally on a wall-mounted panel. The test strips employed are capable of measuring glucose, albumin, urobilin and occult blood. An attached cuff can be used to determine blood pressure, heart rate or temperature. U.S. Pat. No. 5,111,539 describes a similar concept and measured health components, but uses a mechanical slider to dip a preloaded test strip into the urine and a finger cuff to measure blood pressure. U.S. Pat. No. 4,943,416 describes a similar, coin-operated system that captures and transfers urine to a temperature- and humidity-controlled holding tank. Vacuum suction is used to move urine test strips throughout the system and chemical reactions are evaluated with light. U.S. Pat. No. 4,901,736 takes matters a step further by collecting urine mid-stream in a vial which maneuvers into place using a mechanical arm. The vial contains weighted balls for assessing specific gravity and test strips for evaluating specific urine components.

In addition to incorporating urine test strip sampling, testing and reading into the toilet, a variety of other mechanisms for assessing the contents of urine have also been proposed. U.S. Pat. No. 5,730,149 outlines a toilet seat that can be used to add enzymatic urine analysis functionality to an existing toilet. A urine sampling device on a swing arm that extends into the bowl catches a urine sample midair and then retracts into the seat apparatus, where a syringe transfers the sample to a polarographic flow cell for analysis. Depending on the enzymes incorporated into the analyzing apparatus, the seat can be designed to measure a single analyte such as glucose, protein or occult blood.

U.S. Pat. No. 5,073,500 describes a toilet apparatus which measures the concentrations of urinary components based on the specific wavelength-absorbing characteristics of a urine sample following passage through a liquid chromatograph. Urine collected from the toilet bowl is separated into sample aliquots by gas injection, combined with a urinary component-specific reagent, forced through a liquid chromatograph and then exposed to a component-specific wavelength of light. The concentration of the urinary component of interest is calculated based on the relative intensity of the wavelength that successfully passes through the sample to a photodiode. Different urinary components can be assessed by changing the reagents and wavelengths employed by the device.

A different approach is described in U.S. Pat. No. 7,812,312 which describes a system for analysis of aqueous systems using attenuated total reflectance (ATR) crystals. ATR crystals enhance spectral analysis by repeatedly reflecting the sampling beam against the interface between the crystal and the sample, thereby increasing the number of interactions with the sample, improving the signal-to-noise ratio and optimizing the sensitivity, accuracy and speed of the system. In a toilet embodiment of the invention, the ATR apparatus is preferentially incorporated into a separate sampling line branching from the drain pipe of the toilet and with the proposed ability to qualitatively and/or quantitatively to analyze the alcohol, carbon dioxide, creatinine, phosphoric acid, protein, saccharide, triglyceride, and urea content of urine. The system could also be used to assess the fat content of feces. Along the same lines of signal enhancement, U.S. Pat. No. 8,213,007 characterizes a system wherein specific analytes of interest are adsorbed by the nano-structured surface of a chemical sensor, thereby enhancing their Raman signature. Although not a primary embodiment of the invention, it is suggested that the system could be connected to a toilet to facilitate disease and drug detection.

A more feasible use of spectroscopy for toilet-based urine analysis is outlined in U.S. Pat. No. 5,815,260, which describes a toilet stool-based analytical system that measures the concentrations of urogenous components using Raman spectroscopy. Urine is collected in a frontal basin and irradiated by a laser unit preferentially operating at a wavelength of at least 800 nm. The light scattered by the urine passes through a filter to eliminate Rayleigh light so that only the Raman scattered light reaches the photodetector and is analyzed. Spectral results from the sample are compared to a calibrated chemometric model to generate quantitative measurements of urinary components. For example, Dou et al report high correlation coefficients (i.e., R>0.95) for urine samples spiked with acetone, albumin, β-hydroxybutyric acid, creatinine, diautobilirubin, fructose, galactose, globulin, glucose, hemoglobin, lactose, lithium acetoacetate, sodium nitrite, urea and urobilin.

Another practical approach is described in U.S. Pat. No. 5,772,606, which describes an integrated urinal- or toilet stool-based spectroscopic system that analyzes uric component concentrations by measuring urine sample absorbance of select wavelengths of visible or near-infrared light using a rotating filter to selectively expose the sample to a specific set of wavelengths following urine collection in a frontal basin. For example, creatinine is preferentially measured using one or more wavelengths selected from 9,370 to 5,870 cm⁻¹, 5,810 to 5,280 cm⁻¹, 4,980 to 4,730 cm⁻¹ or 4,290 to 4,090 cm⁻¹. Similar sets of preferred wavelength ranges are provided for glucose, hemoglobin, albumin, lithium acetoacetate, ascorbic acid, sodium chloride and sodium nitrite. Absorbance values in these ranges are chemometrically extrapolated using previously generated formulae to provide the concentration of the component of interest.

While the scope of in-toilet urine collection and analysis options proffered over the years illustrates the distasteful nature of urine sampling and testing, prior attempts to automate urine testing face significant limitations, ranging from reliance on consumables to contamination issues to mechanical failure points. These limitations make it difficult to provide the continuous on-site testing necessary to assess individual- and population-level health trends, monitor health changes and ultimately enable health prediction. As a result, although predictive health parameter monitoring systems have been described previously, a truly practical approach has not yet been identified. For example, U.S. Pat. No. 5,073,500 incorporates a data processing system which can compare a user's current results with previous assessments (stored on an individual's integrated circuit card) and provides the user with feedback on changes in component concentration, probable health status, and a prediction of disease state based on changes from previous values or current concentrations of specific urinary components. However, the entire system is liquid chromatography-based, and the regular maintenance required for such a system make it an unworkable option for continuous on-site analysis. In addition, even if the system could be adapted to ongoing use, the small number of urinary components assessed by the system severely limit its diagnostic utility.

A more robust system is detailed in U.S. Pat. No. 7,808,633, which describes a method for using Raman spectroscopy to generate a paired reference database of healthy and diseased biological samples that can subsequently be used with an unknown sample to predict disease progression in an individual, although it makes no provisions for an on-site health analysis system. Spectroscopic data sets for known diseased and healthy samples are compiled into a database that is used as a chemometric reference for unknown samples. For example, after creating a reference database for prostate tissue samples, Maier et al were able to use this database to identify progressive prostate cancer in test samples with 90% sensitivity and 77% specificity. A similar approach using Fourier-transform infrared spectroscopy to assess an individual's disease status from urine and other biological fluids is suggested in U.S. Pat. No. 7,524,681. Unfortunately, both of these systems are designed around the conventional laboratory methodology and face the same single-point data acquisition problems described previously. While the data they generate is useful, it lacks the real-time assessment capabilities facilitated by an on-site, continuous sample acquisition and analysis system.

Accordingly, there is a need to develop a human waste product analysis system that improves on the systems described above by generating longitudinal data derived from frequently acquired data sets.

### SUMMARY OF THE INVENTION

The present invention generally relates to a human health property monitoring system configured to collect and analyze data derived from human waste relating to a property of the human waste. The data comprises longitudinal data having a statistically significant plurality of data sets derived from or corresponding to individual waste samples collected over a period of time and that is sufficient to establish a statistically significant baseline or trend of the one or more property.

In one aspect, the human health property monitoring system comprises:

a human waste receptacle for collecting human waste from a user;

one or more analytical instruments connected to the human waste receptacle and configured to analyze one or more property of the human waste collected by the human waste receptacle;

an electronic storage medium configured to store longitudinal data corresponding to the one or more properties of the human waste, wherein the longitudinal data comprises a statistically significant plurality of data sets corresponding to individual waste samples collected over a period of time sufficient to establish a statistically significant baseline or trend of the one or more property; and

a computer processor configured to determine a statistically significant attribute of the longitudinal data.

In another aspect, the longitudinal data corresponding to the one or more properties of the human waste further comprises a time component selected from one or more of a date, a time and a frequency related to when the human waste was collected.

In another aspect, the human health property monitoring system further comprises an input to receive a user identification corresponding to a source of the human waste. In some embodiments, the user identification corresponds to a single individual. In other embodiments, the user identification comprises demographic information corresponding to an individual. In some embodiments, the demographic information is anonymized. In another aspect, the statistically significant attribute corresponds to data from a single individual.

In another aspect, the longitudinal data comprises a statistically significant plurality of data sets corresponding to individual human waste events over a period of time sufficient to establish a statistically significant deviation for a given individual from an individual baseline, an individual trend, or from a population or sub-population norm. In some embodiments, the statistically significant deviation constitutes a statistically significant pre-symptomatic deviation. In another aspect, the computer processor further comprises a notice routine configured to send an electronic notice of the statistically significant deviation to a designated recipient.

In another aspect, the system further comprises a diagnostic routine configured to send an electronic diagnosis of the statistically significant deviation to a designated recipient.

In another aspect, the analytical instrument comprises a spectrometer.

In some aspects, the computer processor is configured to communicate with a plurality of human waste receptacles.

In some aspects, the plurality of human waste receptacles each comprises an input to receive a user identification corresponding to an individual user who is the source of the human waste, and wherein the data comprises the user identification corresponding to the individual user.

In some aspects, the plurality of human waste receptacles each comprises an input to receive a user identification corresponding to an individual user who is the source of the human waste, wherein the data comprises a plurality of user identifications corresponding to a plurality of users. In some aspects, the plurality of users comprises a sufficient number of users to establish a statistically significant population or sub-population norm. In some aspects of the invention, the data is derived from a sufficient number of users to determine a statistically significant deviation from the population or sub-population norm.

In other aspects, the computer processor further comprises a notice routine configured to send an electronic notice of the statistically significant deviation to a designated recipient.

In some aspects, the data is derived from a sufficient number of users to establish a statistically significant norm of a discrete sub-population group. In some embodiments, the discrete sub-population group comprises a medical practice group, hospital, school, prison or business group.

These and other aspects of the present invention are realized in the present specification and claims, as shown and described in the following figures and related description. It will be appreciated that various embodiments of the invention may not include each aspect set forth above and aspects discussed above shall not be read into the claims unless specifically described therein.

### BRIEF DESCRIPTION OF THE DRAWINGS

The present invention is illustrated and described in reference to the accompanying drawings in which:

FIG. 1(A) is an overhead view of a toilet-based health analysis system depicting the internal arrangement of the various components according to an embodiment.

FIG. 1(B) is a side sectional view of a toilet-based health analysis system depicting the internal arrangement of the various components according to an embodiment.

FIG. 2 is a diagram depicting the system for processing and storing results obtained from the toilet-based health analysis system and the method for assessing, monitoring and predicting the health status of the user, wherein the urinary component concentration calculation is used to identify disease markers, analyze trends and evaluate the overall health status or disease state risk of the user.

FIG. 3 is a diagram depicting the system for processing and storing results obtained from the toilet-based health analysis system and the method for assessing, monitoring and predicting the health status of the user, wherein the user is identified using a urinary fingerprint analysis.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity. Similarly, not every embodiment need accomplish all advantages of the present invention.

### DETAILED DESCRIPTION

The invention and accompanying drawings will now be discussed in reference to the numerals provided therein so as to enable one skilled in the art to practice the present invention. The skilled artisan will understand, however, that the apparatuses, systems and methods described below can be practiced without employing these specific details, or that they can be used for purposes other than those described herein. Indeed, they can be modified and can be used in conjunction with products and techniques known to those of skill in the art in light of the present disclosure. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

Reference in the specification to “one embodiment” or “an embodiment” means that a particular feature, structure, or characteristic described in connection with the embodiment is included in at least one embodiment, but is not a requirement that such feature, structure or characteristic be present in any particular embodiment unless expressly set forth in the claims as being present. The appearances of the phrase “in one embodiment” in various places may not necessarily limit the inclusion of a particular element of the invention to a single embodiment, rather the element may be included in other or all embodiments discussed herein.

Furthermore, the described features, structures, or characteristics of embodiments of the invention may be combined in any suitable manner in one or more embodiments. In the following description, numerous specific details are provided, such as examples of products or manufacturing techniques that may be used, to provide a thorough understanding of embodiments of the invention. One skilled in the relevant art will recognize, however, that embodiments of the invention may be practiced without one or more of the specific details, or with other methods, components, materials, and so forth. In other instances, well-known structures, materials, or operations are not shown or described in detail to avoid obscuring aspects of the invention.

Before the present invention is disclosed and described in detail, it should be understood that the present disclosure is not limited to any particular structures, process steps, or materials discussed or disclosed herein, but is extended to include equivalents thereof as would be recognized by those of ordinarily skill in the relevant art. More specifically, the invention is defined by the terms set forth in the claims. It should also be understood that terminology contained herein is used for the purpose of describing particular aspects of the invention only and is not intended to limit the invention to the aspects or embodiments shown unless expressly indicated as such. Likewise, the discussion of any particular aspect of the invention is not to be understood as a requirement that such aspect is required to be present apart from an express inclusion of the aspect in the claims.

It should also be noted that, as used in this specification and the appended claims, singular forms such as “a,” “an,” and “the” may include the plural unless the context clearly dictates otherwise. Thus, for example, it is understood that a reference to “an engagement element” may include one or more of such engagement elements. In particular, with respect to the construction of claims, it is further understood that a reference to “an engagement element” reads on an infringing device that has more than one engagement element, since such infringing device has “an engagement element”, plus additional engagement elements. Accordingly, the use of the singular article “a,” “an,” and “the” is considered open-ended to include more than a single element, unless expressly limited to a single element by such language as “only,” or “single.”

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

The present invention describes a system and method for assessing, monitoring and predicting disease and/or disease progression through ongoing and longitudinal analysis of various health-related parameters.

The data utilized for purposes of assessing, monitoring and predicting disease and/or disease progression may be obtained using various acquisition mechanisms, for example, any suitable toilet or urinal, or other device designed to capture and analyze human waste may be used. Thus, although the application of such a system is shown in the context of a basic toilet, it should be understood that other configurations are contemplated.

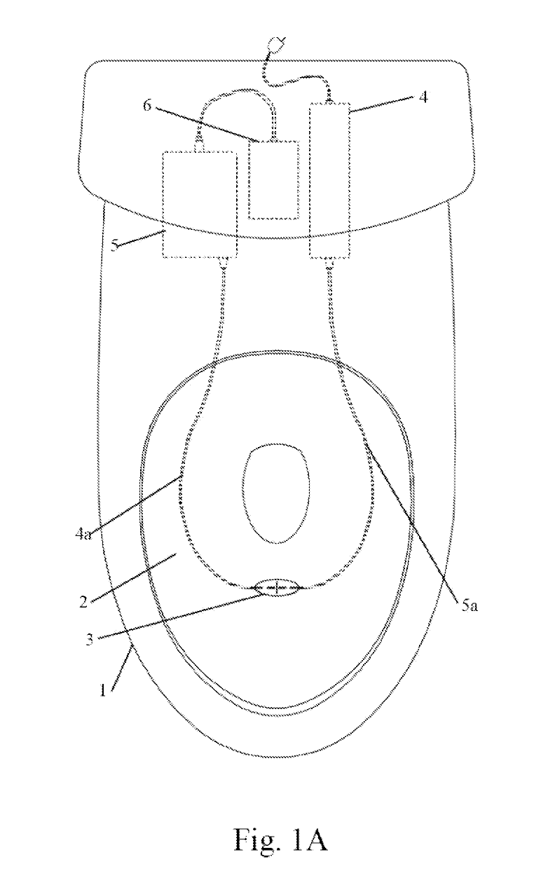

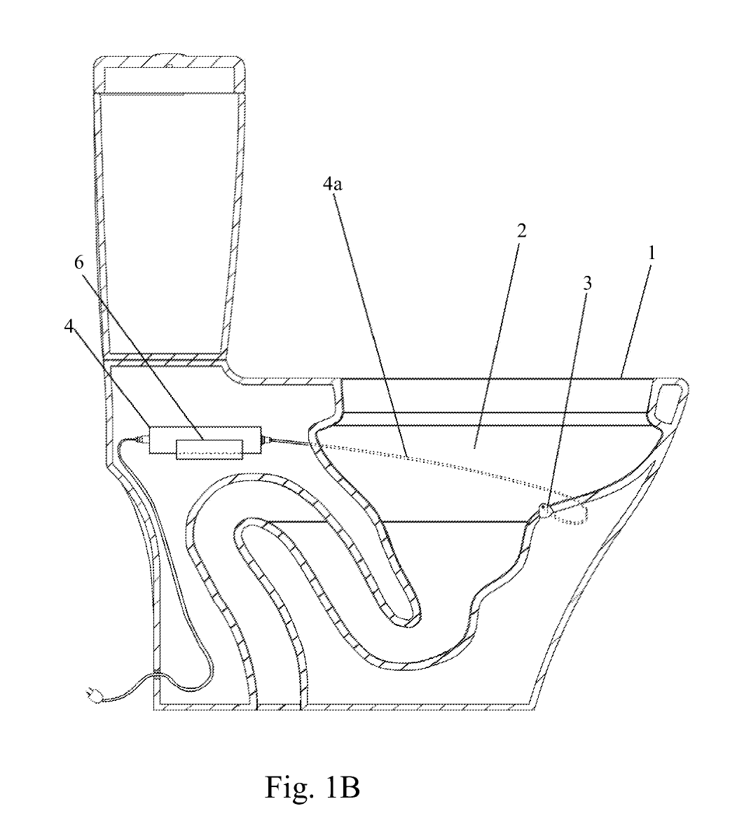

In one embodiment, the system comprises a near-infrared spectrometer integrated into a toilet, for example, as shown in FIG. 1. A unique spectra is obtained for each urine scan and chemometrically extrapolated to determine the concentration of a plurality of urine components. The concentration of these urine components, along with specific changes in the urinary spectra form the basis for a centralized, continuously updated reference database that can be used to assess, monitor and predict health outcomes. New sample spectra and extrapolated concentrations are compared against the reference database using statistical techniques to identify characteristics in keeping with diseased or non-diseased health states. Additionally, sample data is compared on an ongoing basis against the user's own historical results to detect significant changes or trends in health status. By enabling ongoing longitudinal analysis of a broad range of health-related urinary parameters, the toilet-based system can assess, monitor and predict the health of a user.

The present invention further relates to systems and methods for the in vitro detection and evaluation of analytes in human waste, such as urine and/or feces, using one or more analytical tools incorporated into a toilet stool. For example, the toilet stool may employ a Raman spectroscopy system capable of irradiating a sample and producing a Raman spectrum consisting of scattered electromagnetic radiation. Data collected may be processed by an integrated or remote processor to provide information about one or more analytes.

In one aspect, the present invention provides a human health property monitoring system, comprising a human waste receptacle for collecting human waste from a user; one or more analytical instruments connected to the human waste receptacle and configured to analyze one or more property of the human waste collected by the human waste receptacle; an electronic storage medium configured to store longitudinal data corresponding to the one or more property of the human waste, wherein the longitudinal data comprises a statistically significant plurality of data sets corresponding to individual waste samples collected over a period of time sufficient to establish a statistically significant baseline or trend of the one or more property; and a computer processor configured to determine a statistically significant attribute of the longitudinal data.

As described in detail herein, the human health property monitoring system of the present invention provides a significant advance over the prior art with respect to the informational content of data received and processed by the system. In particular, the collection of longitudinal data over a period of time may provide a statistically significant plurality of data sets corresponding to individual waste samples collected over a period of time sufficient to establish a statistically significant baseline or trend of the one or more property.

As used herein, the term “statistically significant” means that a sufficient number of samples is obtained to achieve a confidence level that is statistically meaningful and representative of the condition or state of the user. In scientific terms, a statistically significant result is attained when a ρ-value is less than the significance level. The ρ-value is the probability of observing an effect given that the null hypothesis is true whereas the significance or alpha (α) level is the probability of rejecting the null hypothesis given that it is true. As a matter of good scientific practice, a significance level is chosen before data collection and is usually set to 0.05 (5%). Other significance levels (e.g., 0.01) may be used, depending on the field of study. Statistical significance is used as a measure of the probability of whether or not a data point or set of data points are consistent with a parent data set or fall outside parent data set norms. For example, urine urea values repeatedly acquired over multiple weeks should normally be distributed for a given individual. Based on the normal distribution, 95% of results should fall within two standard deviations of the mean urea value for the individual. For example, if 95% of an individual's urine urea concentrations fall between 1,500 and 2,000 mg/lL, a urine urea concentration of 1,990 mg/dL would not be considered a statistically significantly high value, at a significance level of 5%. However, a urine urea concentration of 2,010 mg/dL would be considered statistically significantly high because the likelihood that the measurement is due to random chance is less than 5%. The measurement is not reasonably explained by random variation. It will be apparent to those skilled in the art that the bounds set for statistical significance will be set in accordance with various parameters; including, but not limited to: odds ratio, relative risk, variability of data, consequences of false positive and false negative results or other relevant considerations. Thus, statistical significance is not limited to results having a ρ-value of less than 0.05%. Accordingly, the importance of any given factor or set of factors will be determined individually and such determinations are known to those skilled in the art as described, for example, by Munro, B., Statistical Methods for Health Care Research (Lippincott Williams & Wilkins, 2005. In accordance with such guidelines, those skilled in the art may select a ρ-value threshold to less than 0.05%, for example, 0.04%, 0.03%, 0.02% or 0.01%.

The term “longitudinal” means data that has been acquired over a period of time and represents a plurality of data points obtained at different times over such period of time, for example, days, weeks, months, or years. Longitudinal data may include, for example, data for a variety of trends and/or patterns, including, but not limited to, cyclic structures, periodicity, changes in levels over time as indicators of changing health condition, and/or changes in variability over time as indicators of changing health condition.

In some embodiments of the human health property monitoring system of the present invention, the longitudinal data corresponding to the one or more properties of the human waste may further comprises a time component selected from one or more of a date, a time and a frequency related to when the human waste was collected. The time component of the data may indicate, for example, a date, a time of day, a season of the year, a year, etc. so that the data can be tracked chronologically and used to evaluate historical patterns of the patient's health condition and predict future health conditions based on extrapolation of historical data.

It is further contemplated that in some embodiments the human health property monitoring system of the present invention may further comprise an input to receive a user identification corresponding to a source of the human waste. The user identification will ordinarily correspond to a single individual, and may comprises patient identifying information and non-identifying information, such as demographic information. As described in further detail herein, the demographic information may be anonymized to protect the identity of the user.

In some aspect of the human health property monitoring system of the present invention, the statistically significant attribute corresponds to data from a single individual. It is contemplated, for example, that a single individual will utilize the system of the present invention frequently, for example, multiple times per day, daily, multiple times per week, so as to obtain a set of data representing the health condition of the user over a sufficiently long period of time, with a sufficient number of data points, that it is possible to establish a statistically significant base line or trend that reflects the health condition of the user. The baseline may represent a healthy condition, from which a deviation represents a non-healthy condition. Alternatively, the baseline may represent a non-healthy condition, from which the deviation represents a return to a healthy condition. Thus, in some embodiments, the longitudinal data comprises a statistically significant plurality of data sets corresponding to individual human waste events over a period of time sufficient to establish a statistically significant deviation from a baseline, a trend, or from a population or sub-population norm.

The system of the present invention may also be used to detect and analyze non-health conditions or disease states based on chemical variations or deviations prior to such variations or deviations presenting symptoms that are discernible to the user. Thus, in some embodiments, the statistically significant deviation constitutes a statistically significant pre-symptomatic deviation. It is further contemplated that in some embodiments, the statistically significant deviation constitutes a statistically significant post-symptomatic deviation, or a deviation indicative of the future progression of a health or disease state.

In some embodiments, it is desirable that the human health property monitoring system be configured to notify health care professionals or the user of changes in health status that may be important to the health of the user. Thus, in some embodiments, the system may further comprise a diagnostic routine configured to send an electronic diagnosis of the statistically significant deviation to a designated recipient. In other embodiments, the computer processor further comprises a notice routine configured to send an electronic notice of the statistically significant deviation to a designated recipient. Many disease states, for example, demonstrate improved response to drug treatments when initiated earlier in the disease state. Accordingly, an early warning system may be useful in developing more effective treatment regimens.

In accordance with the present invention, the computer processor may be configured to communicate with a plurality of human waste receptacles. In some embodiments, the plurality of human waste receptacles are electronically and communicatively connected, thereby enabling an individual user's data to be collected from the plurality of human waste receptacles (i.e., one at work, another at home, another in an airport, etc.) and pooled into a single data system so as to increase the number and frequency of relevant data points and thereby increase the power and accuracy of the data to establish a norm or trend away from the norm.

Accordingly, in some embodiments, each of the plurality of human waste receptacles each may comprise an input to receive a user identification corresponding to an individual user who is the source of the human waste. When a user uses a particular waste receptacle, the waste receptacle may be configured to identify the user and correlate the user with the data corresponding to that user's waste analysis. Similarly, some waste receptacles that are used by more than one user may be configured to identify more than one user. Accordingly, in other embodiments the plurality of human waste receptacles each comprises an input to receive a user identification corresponding to an individual user who is the source of the human waste, wherein the data comprises a plurality of user identifications corresponding to a plurality of users.

Where the human health monitoring system of the present invention is used to collect data from a plurality of users, it is possible then to track data corresponding to the plurality of users, for example, a group of individuals in a common home, common work environment, common hospital, common zip code, common city, common geographical region, etc., which would enable the system to identify norms and trends in such population or sub-population, or as compared to other populations or sub-populations. Accordingly, in some embodiments, the present invention provides a human health property monitoring system, wherein the plurality of users comprises a sufficient number of users to establish a statistically significant population or sub-population norm. In other embodiments, the data is derived from a sufficient number of users to determine a statistically significant deviation from the population or sub-population norm. For example, in some embodiments, the human health property monitoring system of the present invention may track data from a discrete sub-population group comprising a single home, a medical practice group, hospital, school, prison or business group. Sub-populations could also include, for example, sub-populations defined according to age, blood glucose, body-mass index, current and past medications, diagnoses of a particular disease, dietary patterns, elevation, gender, general geographic location, height, independent laboratory results, medical diagnostic test results, medical history, race, temperature, wearable device results, weight, or any other relevant factor related to health or disease states.

In some embodiments, the computer processor further comprises a notice routine configured to send an electronic notice of the statistically significant deviation to a designated recipient. In other embodiments, the data is derived from a sufficient number of users to establish a statistically significant norm of a discrete sub-population group.

FIG. 1(A) and FIG. 1(B) depict a toilet-based health analysis system which can be used to quantify the concentrations of a multiplicity of urinary components in an automatable, reagent-free manner which is readily amenable to domestic or other on-site environments, thereby allowing for acquisition of the continuous measurements necessary to assess, monitor and predict the health status of the user.

A toilet body 1 has a toilet bowl 2, a urine sampling device 3, a light source part 4, a light measuring part 5, and a computing and transmitting part 6. In the depicted embodiment, the urine sampling device 3 which is integrated into the toilet bowl 2 is provided with a urine sampling cell 6, such that urine flowing across the toilet bowl 2 passes over the urine sampling device 3 and through the urine sampling cell 6. The urine sampling cell 6 contains a thermistor for detecting when urine has been introduced into urine sampling cell 6 by means of a temperature change resulting from the presence of urine. In some embodiments, the thermistor may detect a specific range of temperature consistent with a normal body temperature of a user, for example, ranging from about 90° F. to about 106° F., or alternatively from about 97° F. to about 100° F., or alternatively from about 97.7° F. to about 99.5° F.

A light source part 4 is provided for irradiating the urine sample cell 6 with a measuring beam, while a light measuring part 5 is provided for receiving and detecting the measuring beam transmitted through the urine sampling cell 6. The measuring beam is conducted from the light source part 4 to the urine sample cell 6 through a light emitting fiber 4a and is conducted from urine sample cell 6 to the light measuring part 5 through a light receiving fiber 5a. The light source part 4 and the light measuring part 5 serve both as means for measuring absorbances of a urine sample in the urine sampling cell 6 at the urine sampling device 3 and as a sensor for detecting soiling of the urine sample cell 6 by measuring changes in the absorbance of the cell itself in order to determine the degree of soiling of the urine sample cell 6.

The light source part 4 comprises a lamp source emitting light of a continuous range of wavelengths, a light-emitting diode array emitting light of a continuous range of wavelengths, a laser unit having a variable oscillation wavelength, or a laser diode array emitting laser beams of measuring wavelengths. The light measuring part 5 is provided with a spectrometer component or interferometer component and a photodetector component comprised of a photodiode, an array type photoreceptor of CCD, a photoreceptor array or a single photoreceptor as a detector. Light intensity or quantity measurement sensitivity depends on optical path lengths and wavelengths. The urine sample cell 6 is not restricted to a single optical path length, but can be provided with continuously or step-wisely differing optical path lengths chosen in a manner that optimizes the signal-to-noise ratio for a given wavelength or set of wavelengths. Additionally, measuring time may be used to improve signal-to-noise ratio for a given wavelength, set of wavelengths, or the spectra as a whole and may be chosen from the time range of 10 to 1,800,000 ms. Following emission from the light emitting fiber 4a, the measuring beam is transmitted through the urine sample cell and is received by the light receiving fiber 5a, so that the measuring beam transmitted through the cell is spectroscopically analyzed by the spectrometer component of light measuring part 5 and thereafter guided to the photoreceptor component of light measuring part 5.

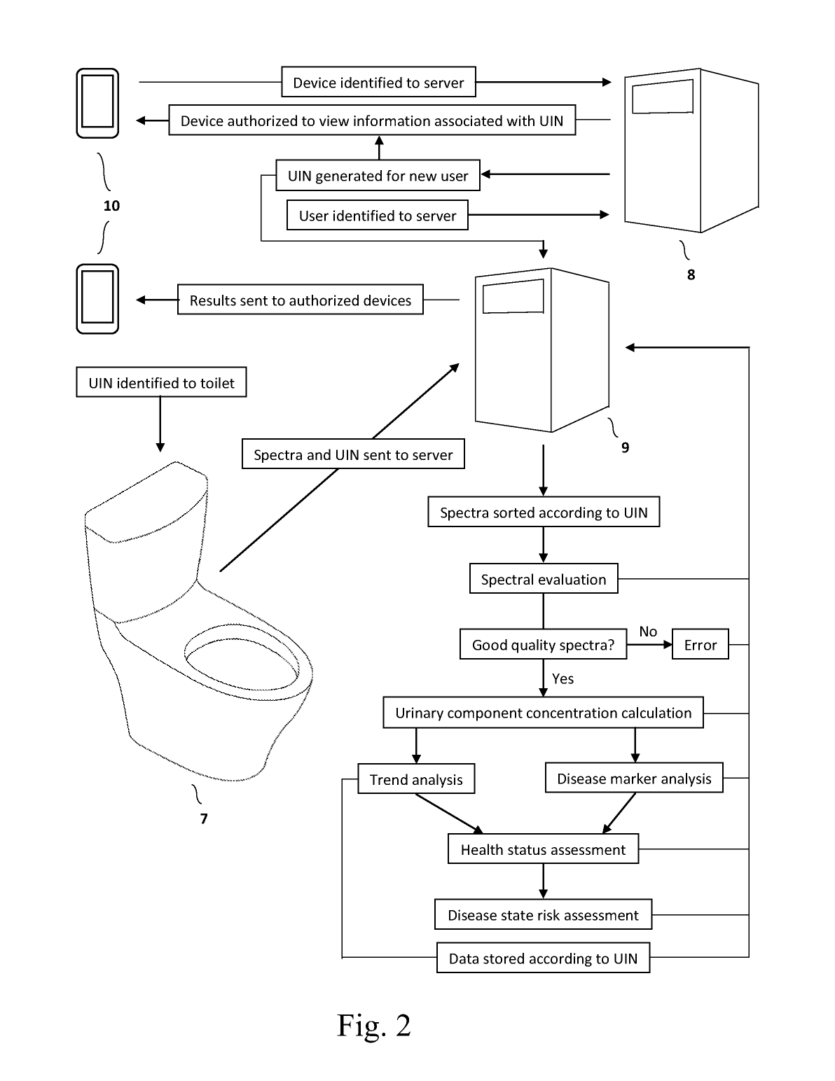

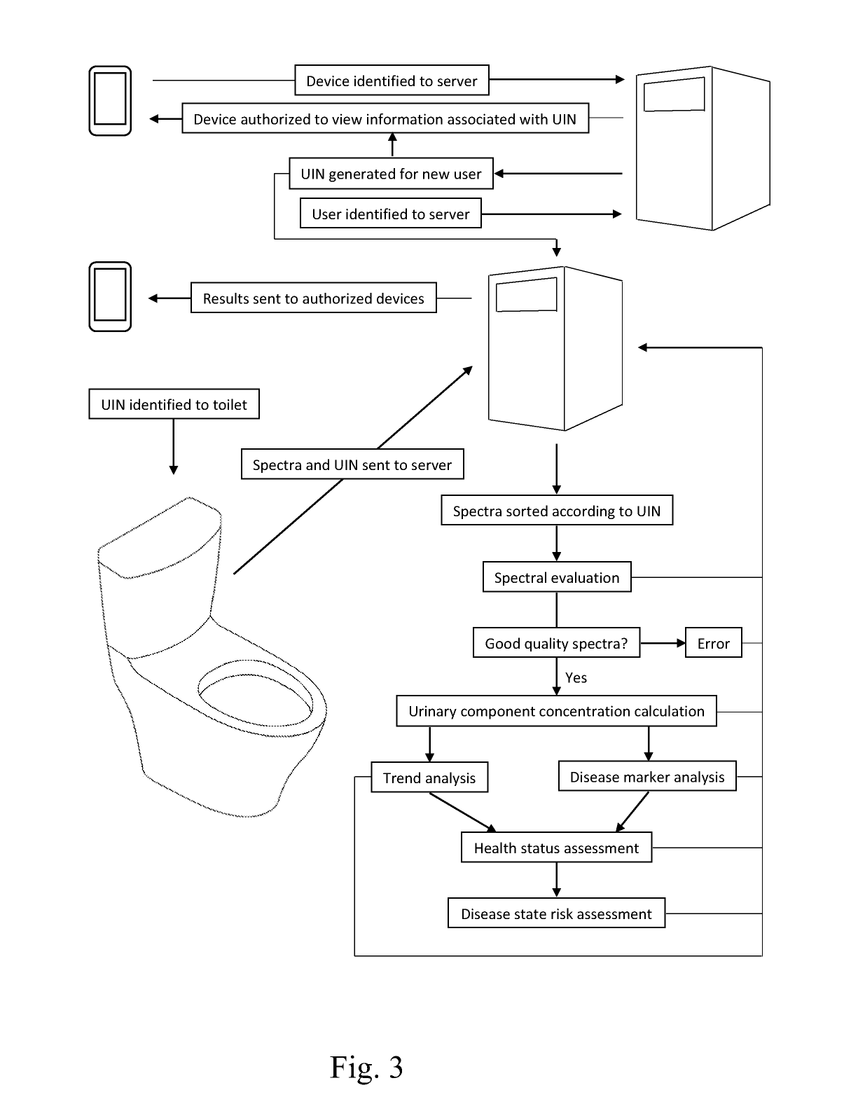

FIG. 2 and FIG. 3 illustrate the system by which absorbance data is transmitted, stored and interpreted, thereby providing continuous health assessment, monitoring and prediction for the user. The system comprises the elements of a toilet body 7, a remote identifying information server 8, a remote data storage and analysis server 9, and an electronic computing device 10 owned and maintained either by the user or a party authorized by the user to receive their health-related information.

Individually identifiable information such as name, address, billing information, or date of birth is stored on the remote identifying information server 8 for each unique user and each unique user is assigned a unique identification number (UIN). Other user-related data may also be associated with the UIN, including gender, race, nationality, socioeconomic status, residential zip code, veteran status, disease biomarker status, etc., which data may be useful in interpretation of population or sub-population studies. This UIN is communicated to the remote data storage and analysis server 9. Additionally, an electronic computing device 10 or multiple devices may be authorized by the user to receive their health-related information. This electronic computing device 10 receives a digital authorizing certificate from the remote identifying information server 8 allowing the electronic computing device 10 to retrieve health-related information associated with the user.

Health state assessment, monitoring and prediction is initiated when a user is identified to the toilet body 7. This identification may occur using a variety of means such as direct entry of the UIN via a built-in, wired, or wireless keypad; wireless pairing with an authorized electronic computing device 10; recognition of implanted, worn or carried radio frequency identification; or fingerprint, retinal scan or other biometric identification. The subsequent urine sample spectra obtained using the toilet body 7 are then coupled with the supplied UIN and wirelessly transmitted to the remote data storage and analysis server 9.

Following receipt of new data from the toilet body 7, the remote data analysis server 9 sorts the spectra data in accordance with the accompanying UIN. Spectra are then evaluated to determine whether or not they meet basic quality parameters. Spectra of sufficient quality undergo algorithmic processing on the basis of the absorbances measured in the toilet body 7 to obtain urinary component concentrations. Spectra of insufficient quality are designated as erroneous and recorded as such. In order to measure a multiplicity of urinary components, measuring wavelengths are selected which are best correlated with urinary component concentrations as measured by a preexisting assay. Wavelengths or wavelength regions having absolute values of correlation coefficients of at least 0.4 to a chosen urinary component are regarded as measuring wavelength regions and are selected from the 100 nm to 4,000 nm wavelength range. Additionally, wavelengths or wavelength regions having absolute values of correlation coefficients of at least 0.1 to the presence, absence or severity of the disease, disease state, health risk factor or other health state are regarded as measuring wavelength regions and are selected from the 100 nm to 4,000 nm wavelength range.

Urinary component concentrations are then evaluated by the remote data analysis server 9 and classified as “normal” or “abnormal.” The remote data analysis server 9 compares the most recently obtained data associated with a UIN with historical data associated with the same UIN to establish trends over time. Urinary components for an individual which have an overall regression slope of less than 0.2 measurement units per time unit, as measured across multiple appropriate time intervals, are defined as “normal” for that individual. Urinary components which have an overall regression slope of 0.2 measurement units per time unit or greater, as measured across multiple appropriate time intervals, are defined as “abnormal” for that user. The remote data analysis server 9 also assesses urinary component results to determine if results are direct markers of disease, disease state, health risk factor or other health state as determined by predefined minimum or maximum healthy values for a healthy individual.

The aggregate of trend analysis and disease marker analysis is then employed by the remote data analysis server 9 to determine the current health status of the user. Changes in trend or disease state markers or in the health status of the user are then used to evaluate the risk that the user will develop a particular disease state within a given time frame. These changes and their significance may identified using a variety of statistical techniques such as partial least squares or principal component regression, although a variety of other techniques may be employed; including, but not limited to: artificial neural networks, multiple linear regression, multivariate curve resolution, support vector machine classification or regression or cluster analysis. Alternatively, machine learning or statistical techniques familiar to those skilled in the art may be employed to identify other predictive aspects derived from continuous monitoring of urine samples. Non-component-specific changes in the urinary spectra may also be evaluated as predictors of changes in components of bodily fluids other than urine or general changes in health status. These predictors have absolute correlation coefficient values between changes in urinary spectra and changes in bodily fluid components or health conditions of at least 0.2. This analysis may be accomplished by the remote data analysis server 9 concurrent with the evaluation of spectral quality.

Following data analysis, the remote data analysis server 9 stores spectral quality and analysis results, urinary component concentrations, trend and disease marker results, health assessment findings, and disease risk results in accordance with their associated UIN. These results may then be accessed by an electronic computing device 10 authorized to view data associated with the appropriate UIN. A rules engine for determining which parameters dictate transmission of an alert to an authorized electronic computing device 10 may be defined on the authorized electronic computing device 10.

In some embodiments of the present invention, measurements collected from the sampling site are communicated wirelessly to a remote server for processing and storage. Each user is assigned a unique identification number (UIN) that pairs spectral data from a given urine or fecal sample with the individual who produced the sample. The system identifies an individual by one of several alternative means; including, but not limited to: direct entry of the UIN via a built-in, wired, or wireless keypad; wireless pairing with a user-owned cellular device; recognition of implanted, worn or carried radio frequency identification; or fingerprint, retinal scan or other biometric identification.

Once the user and their associated UIN have been identified, the UIN is used to link spectra, predicted urinary or fecal component concentrations and other non-identifying health information related to a specific user. Non-identifying health information may include, but is not limited to: age, blood glucose, blood pressure, body-mass index, current and past medications, diagnoses, dietary patterns, gender, general geographic location, height, independent laboratory results, medical diagnostic test results, medical history, race, temperature, wearable device results or weight. This information can be electronically communicated to the server directly by the user or their healthcare provider. Alternatively, the server may be linked to the user's patient file, electronic health record or other medical database, allowing for online communication of health data. Information may also be added from an independent device used to track the previously described elements or to facilitate documentation of other health-related parameters.

A separate server is used to store individually identifiable information such as name, address or billing information in coordination with the user's UIN. This server issues digital certificates of authorization to the computer, smart device or other electronic devices of the user or another individual or group authorized by the user. These certificates authorize the electronic device to retrieve personal health information associated with the authorized UIN from the previously mentioned remote server. As a result, breach of a single server will not provide both individually identifiable and health information.

Once spectral data has been assigned to the proper user, values at specific points are algorithmically extrapolated to generate the predicted concentrations of urinary or fecal components in a sample or to identify the presence, absence or severity of a disease, disease state, health risk factor or other health state for the sampled individual. To avoid faulty data, scans may be discarded if values at specified points lie outside predetermined minimums and maximums. Results are stored as previously mentioned and all results are preferably plotted as a time series. Since all possible algorithmic extrapolations may not be identified prior to sampling, stored spectra may also be retroactively reprocessed using algorithms developed subsequent to sample acquisition to determine historic concentrations of urinary or fecal components in one or more samples or to identify the historic presence, absence or severity of a disease, disease state, health risk factor or other health state for the sampled individual. In addition to the other health-related information elucidated by the proposed invention, the ability to retroactively assess samples for previously unidentified health changes provides a heretofore impossible means for following the course of disease and health.

Data assignation, extrapolation and sequencing allow health parameters present in urine to be tracked and monitored in real-time. This offers numerous advantages over current methodology. First, daily or multi-daily tracking of urinary or fecal components can be used to identify a user's true normal range over time. Currently, test results from a single point in time are used to determine an individual's relative health; however, Knuiman et al (1986) reported in Human Nutrition Clinical Nutrition, 40, 343-348 that it required 4-14 days of continuous 24-hour sampling to estimate urinary components to within 20% of habitual excretion. Knuiman et al (1988) reported similar results in Clinical Chemistry, 34, 135-138, with the added observation that it required 11-26 days, depending on the specific urinary component, of sequential overnight urine sampling to accurately estimate urinary components to within 20% of habitual excretion. Overnight urine testing is far more similar to the routine sampling protocols employed by the medical profession than 24-hour sampling; therefore, since the within-person variability reported in this study ranged from 33-52% for overnight testing, the current inability to acquire numerous sequential samples means that the single-point test results used by healthcare professionals to monitor and treat an individual's health are poor estimates of that individual's typical urinary component concentration. This highlights the utility of the proposed innovation, which, by eliminating the difficulties of conventional urine and fecal testing, makes accurate assessment of an individual's urinary or fecal component concentrations routinely achievable through ongoing monitoring.

Second, daily or multi-daily tracking of urinary components can be used to identify “normal” and “abnormal” trends in urinary or fecal component concentration. Given the human body's predilection to maintain homeostasis, a regression line plotted across the sequential urinary concentrations of various components has an effective slope of zero, given an appropriate time window. There may be a sinusoidal component to the production and/or excretion of certain urinary or fecal components which may follow circadian, diurnal, nocturnal, monthly or other biologic rhythms; however, the overall slope across multiple cycles for these components remains approximately zero under stable health conditions.

In the present invention, urinary or fecal components for an individual which have an overall regression slope of less than 0.2 measurement units per time unit, as measured across multiple appropriate time intervals, are preferentially defined as “normal” for that individual. This may or may not be substantively different than the normal for the population as a whole. In contrast, an individual's urinary or fecal components which have an overall regression slope of 0.2 or greater measurement units per time unit, as measured across multiple time intervals, are preferentially defined as “abnormal” for that individual. In this way, the proposed invention can be used to identify consistent changes in health, regardless of the presence or absence of symptoms. Whether positive or negative, these changes in excretion represent changes in the fundamental health processes of the user.

Third, disease markers change in advance of observable symptoms; therefore, daily or multi-daily tracking of urinary or fecal components enables pre-symptomatic diagnosis and treatment. For example, kidney stones form subsequent to well-defined changes in urinary components. The solubility of calcium oxalate—the key precipitate in 80% of nephrolithiasis cases—in water is about 0.44 mg/dL; however, this is mitigated by the presence of citrate, which complexes with free calcium ions and inhibits the formation of calcium oxalate crystals. Kidney stones frequently form when the urinary concentration of oxalic acid is consistently above 0.44 mg/dL and citric acid excretion is below 325 mg/24 h. Since the crystallization of renal calculi takes time, continuous monitoring of these urinary components can be used to identify patients at significant risk for kidney stones before the condition becomes symptomatic. Dietary or medical interventions can then be implemented to reverse the crystallization process, allowing the individual to return to a healthy state and circumvent the discomfort of passing a kidney stone. While these predisposing changes in urinary component concentrations have been known for decades, current medical testing is unable to supply the real-time monitoring needed to pre-symptomatically identify and treat nephrolithiasis. This example is representative of many other disease states in which symptoms are preceded by changes in urinary or fecal component concentrations; however, without a system for continuous monitoring of these components, these changes are typically only used in confirmatory testing after symptoms have developed.

Fourth, daily or multi-daily tracking of urinary or fecal components can be used to identify new links between changes in urinary component concentration and the development, progression or exacerbation of a disease state. For example, Loureiro et al (2014) reported in the Journal of Allergy and Clinical Immunology, 133, 261-263 that principal component analysis of urine component concentrations revealed that threonine, alanine, carnitine, trimethylamine-N-oxide and acetylcarnitine concentrations increased and acetate, citrate, malonate, phenylacetylglutamine dimethylglycine and hippurate concentrations decreased during asthma exacerbations. Loureiro et al concluded from their findings that changes in these or other urinary components could be used to predict the onset of an asthma exacerbation. Similarly, Liang et al (2009) reported in Guang Pu Xue Yu Guang Pu Fen Xi, 29, 1772-1776 that Bayes stepwise integration of NIR spectra enabled them to correctly identify chronic enteritis in alpine musk deer with 100% accuracy and identify healthy specimens with 93.3% accuracy. These examples are representative of many other disease states which effect metabolic changes that can be monitored in the urine or feces.

In addition to finding new correlations between disease states and alterations in urinary or fecal component concentrations, continuous monitoring of urinary or fecal spectra can be used to identify wavelengths or groups of wavelengths that vary consistently in accordance with changes in an individual's health condition or the molecular makeup of other body systems or fluids. For example, Purnomoadi et al (2000) reported in Near-Infrared Spectroscopy: Proceedings of the 9th International Conference, 729-733 a correlation coefficient of 0.96 between a urinary absorbance peak located at 2134 nm and the blood urea nitrogen of cows. This wavelength remained highly predictive when the cows' blood urea nitrogen increased in response to stress. Thus, the continuous monitoring provided by the present invention can be used to identify changes in health either directly through urinary component quantification or indirectly through changes in the urinary spectra.

In the preferred embodiment, correlations between changes in urinary or fecal spectra and changes in bodily fluid components or health conditions of at least 0.2, preferably 0.6, are identified using partial least squares or principal component regression, although a variety of other techniques may be employed; including, but not limited to: artificial neural networks, multiple linear regression, multivariate curve resolution, support vector machine classification or regression or cluster analysis. Alternatively, machine learning or other statistical techniques familiar to those skilled in the art may be employed to identify other predictive aspects derived from continuous monitoring of urine or fecal samples.

Lastly, changes in urinary or fecal spectra can be used to monitor drug usage and metabolism. The vast majority of drugs and their metabolites are excreted to some extent in urine and virtually all drugs and their metabolites not excreted in urine are excreted fecally. Thus, by continuously monitoring urine and/or feces, it is possible to determine drug usage and metabolism. Currently, the cost of monitoring drug usage and metabolism is prohibitively time-consuming and expensive. Since drug usage and metabolism are crucial to therapeutic decision-making, the proposed invention offers an unprecedented way to rapidly determine the usage and efficacy of a given drug regimen. Moreover, it also enables affordable and continuous monitoring for illicit drug usage, thereby both improving compliance with prescribed drugs and constraining misuse of drugs.

The present invention may include a combination of one or more analytical tools with their associated reagents and any variants or new and/or alternative analytical techniques designed for use with those tools as recognized by those skilled in the art of laboratory analysis, including, but not limited to: Raman spectrometer, nuclear magnetic resonance (NMR) spectrometer, near infrared (NIR) spectrometer, infrared spectrometer, ultraviolate spectrometer, visible light spectrometer, gas chromatograph (GC), liquid chromatograph (LC), high performance liquid chromatograph (HPLC), mass spectrometer (MS), microscope, photographic camera, ion fuel-cell devices, ion-selective electrode, weight scale, Geiger counter, thermometer, pH gauge, flowmeter, colorimeter, enzyme electrode, enyzme-linked immunosorbent assay (ELISA), color sensor, test strips, oxidation-reduction reagents, precipitants, magnetometer, photometer, microbial growth media, refractometer, antibodies, and other reagents. Sampling for these tools, which are preferentially positioned within the toilet, may occur at one or more sites in or on the toilet bowl and/or piston chamber.

In one embodiment, spectroscopic components may produce radiation and provide spectroscopic measurements of a urinary and/or fecal sample. For example, an 805 nm, focusable 800 mW laser may be directed to a sample through a 50/50 beam splitter and a microscope objective lens. The light is then preferentially passed through a notch filter, 50 μm slit, and plano/convex lens before it is focused onto a holographic diffraction grating to produce a spectrum. The resulting spectrum is directed to a charged coupled device (CCD), generating a spectral image which may then be translated into a Raman signature using analytical software.

In another embodiment, microscopic components may produce radiation and provide microscopic images of a urinary and/or fecal sample. For example, light may be emitted from a directed illumination source through a condenser annulus and focused on a sample by a condenser. Light scattered by the specimen and background light may be focused through an objective lens and may then be passed through a phase shift ring and a gray filter ring to improve the contrast between the scattered light and the background light. The final image may then be captured by a digital camera for computerized assessment and/or storage.

In another embodiment, a thermometer, pH gauge, ion-selective electrode, and/or enzyme electrode, either positioned within the device or embedded in a surface of the device, are exposed to or extended into urine and/or feces. Weight scales and/or flowmeters, preferably positioned on one or more surfaces of the device, may also be included to provide measurements of the sample or total urine and/or fecal volume. An alcohol-sensitive ion-fuel cell device may also be placed above a urine collection and/or sampling site for collection and/or analysis of urine and/or fecal vapors. A digital camera may also be placed under the seat or within the piston chamber to photograph specimens.

In another embodiment, a pulse sensor, oxygen saturation monitor and/or bioelectric impedance analyzer, may be preferentially placed on and/or within a toilet and in contact with a user's skin for measurement of various physiological parameters. Additionally, a body weight assessment tool, such as a pressure-sensitive film placed under the seat, may be included to assess the user's weight.

In another embodiment, one or more reagent, precipitant, antibody and/or other additive (collectively referred to in this paragraph as “reagent(s)”) reservoirs may dispense a measured quantity of reagent(s) into a urine and/or fecal sample, where an agitating device may be used to ensure even dispersion of the reagent(s) within the sample. Depending on the reagents employed, the resulting mixture may be the subject of subsequent sampling and/or analysis as outlined in other descriptions of potential embodiments of the invention.

In addition to these embodiments, a toilet may feature other analytical tools. For example, one or more test strip containers may be used to dispense one or more test strips to a user or insert one or more test strips into a sample. Depending on the test strips employed, the strips may be the subject of subsequent analysis as outlined in other descriptions of potential embodiments of the invention. The apparatus may also contain one or more microbial growth media reservoirs which dispense a measured quantity of growth media into a sample of urine and/or feces, where an agitating device may be used to ensure even dispersion of the sample within the growth media. The resulting mixture may then be incubated for an appropriate amount of time, whereupon it may be subject to subsequent sampling and/or analysis as outlined in other descriptions of potential embodiments of the invention. Geiger counters, refractometers, colorimeters, photometers and/or magnetometers may also be included to provide measurements of the urine and/or fecal sample and/or total urine/fecal volume.

The present invention may be used to obtain a wide range of information about the physical and/or chemical properties of a user's urine and/or feces. For example, a toilet may provide information about one or more urine and/or fecal analytes, their metabolites and/or related biomarkers; including, but not limited to: amino acids; antioxidants, cancer biomarkers, catecholamines, cholesterol synthesis biomarkers, disease state biomarkers, environmental toxins, enzymes, ethanol, hormones, inflammatory biomarkers, prescription or over-the-counter drugs, illicit drugs, metabolic products, microbial biomarkers, minerals, and/or oxidative stress biomarkers. The device may also provide information about one or more other aspects of the user's urine and/or feces; including, but not limited to: casts, crystallization, density, fat content, fiber content, microbial content, protein content, radioactivity, red blood cell count, specific gravity, temperature, vitamin content, and/or white blood cell content. Additionally, the apparatus may provide information regarding other aspects of the user's physical and/or physiologic state; including, but not limited to: body weight, body mass index, bioelectric impedance, body fat content, oxygen saturation and/or pulse rate. This information may be used independently, to directly replicate standard clinical laboratory tests or as surrogate markers for analytes typically used in standard laboratory tests.

In one aspect of the invention, a toilet may be used to detect the presence and/or concentration of one or more metabolic products in urine and/or feces. Since well over 3,100 metabolites have been identified in urine alone, only a small fraction of the metabolic analytes that may be assessed are listed in Table 1. These metabolites may be the result of amino acid metabolism, antioxidant metabolism, cancer metabolism, cholesterol synthesis, disease activity, enzymatic action, hormone synthesis and metabolism, inflammation, microbial metabolism, oxidative stress or other metabolic processes.

In another aspect of the invention, a toilet may be used to detect the presence, type, and/or quantity of one or more types of microbes in urine and/or feces. The toilet may also be used to detect the presence, type and/or quantity of one or more types of human cells in urine and/or feces; including, but not limited to: epithelial cells, red blood cells and/or white blood cells.

TABLE 1
Metabolomic Analytes.

| | | |
|---|---|---|
| Amino Acids | Organic Acids | Hormones |
| 1-methylhistidine | 2-OH-phenylacetic acid | Cortisol |
| 3-methylhistidine | 3-OH-propionic acid | Dehydroepiandrosterone |
| α-aminoadipic acid | 4-OH-phenylpyruvic acid | Estradiol |
| α-aminoisobutyric acid | α-ketoadipic acid | Estriol |
| Alanine | α-ketoisocaproic acid | Estrone |
| Ammonia | α-ketoisovaleric acid | Growth Hormone |
| Anserine | α-keto-β-methylvaleric acid | Human Chorionic Gonadotropin |
| Arginine | Formiminoglutamic acid | Pregnanediol |
| Asparagine | Glucaric acid | Progesterone |
| Aspartic acid | Homogentisic acid | Testosterone |
| β-alanine | Kynurenic acid | Thyroxine |
| β-aminoisobutyric acid | Methylmalonic acid | Triiodothyronine |
| Carnosine | Orotic acid | Cancer Biomarkers |
| Citrulline | Pyroglutamic acid | 5-hydroxyindoleacetic acid |
| Cystathionine | Catecholamines | β-2-microglobulin |
| Cysteine | Dopamine | β-human chorionic gonadotropin |
| Ethanolamine | Epinephrine | Cyclic adenosine monophosphate |
| γ-aminobutyric acid | Norepinephrine | Chromosome 3 |
| Glutamic acid | Glucose Metabolism | Chromosome 7 |
| Glutamine | Glucose | Chromosome 17 |
| Glycine | Ketones | Chromosome 9p21 |
| Histidine | Others | Fibrin/Fibrinogen |
| Homocysteine | Arabinitol | Homovanillic acid |
| Hydroxylysine | Citric acid | Immunoglobulins |
| Hydroxyproline | Creatinine | Nuclear matrix protein 22 |
| Isoleucine | Diautobilirubin | Antioxidants |
| Leucine | Hippuric acid | 4-hydroxynonenal |
| Lysine | Hydroxybutyric acid | p-hydroxyphenyllactate |
| Methionine | Lactate | L-threonic acid |
| Ornithine | Laurate | Malondialdehyde |
| Phenylalanine | Mannitol | γ-Tocopherol |
| Phosphoethanolamine | Nitrites | Oxidative Stress Biomarkers |
| Proline | Oxalic acid | 8-hydroxydeoxyguanosine |
| Sarcosine | Phylloquinone | Lipid hydroperoxides |
| Serine | Uric acid | Isoprostanes |
| Taurine | Urea nitrogen | Conjugated dienes |
| Threonine | Urobilin | |
| Tryptophan | | |
| Tyrosine | | |
| Valine | | |

In another aspect of the invention, a toilet may be used to detect the presence and/or concentration of one or more minerals in urine and/or feces; including, but not limited to: calcium, chloride, iodine, iron, lithium, magnesium, phosphorus, potassium and/or sodium. The apparatus may also be used to detect the presence and/or concentration of one or more environmental toxins in urine and/or feces; including, but not limited to: aluminum, arsenic, bismuth, cadmium, chromium, cobalt, copper, ethyl benzene, fluoride, lead, manganese, mercury, nickel, phenols, selenium, styrene, thallium, toluene, xylenes, and/or zinc. In another aspect of the invention, a toilet may be used to detect the presence and/or concentration of one or more non-scheduled prescription or over-the-counter drugs and/or their metabolites in urine and/or feces; including, but not limited to drugs classified as: antiarrhythmics, antibiotics, anticholinergics, anticoagulants, anticonvulsants, antidepressants, antihistamines, anti-hyperlipidemics, antihypertensives, antineoplastics, antipsychotics, cortico steroids, immuno suppressants, muscle relaxants and/or non-steroidal anti-inflammatories. In another aspect of the invention, a toilet may be used to detect the presence and/or concentration of one or more scheduled prescription drugs; including, but not limited to drugs classified as: amphetamines, anabolic steroids, barbiturates, benzodiazepines and/or narcotics or opiates. In another aspect of the invention, a toilet may be used to detect the presence and/or concentration of one or more illicit drugs; including, but not limited to: cocaine, heroin, lysergic acid diethylamine, marijuana, phencyclidine, or other illicit drugs classified as: barbiturates, benzodiazepines, hallucinogens, hypnotics, narcotics, stimulants and/or synthetic cannabinoids. In another aspect of the invention, a toilet may be used to detect the presence, type, and/or quantity of specific foods and/or dietary components in urine and/or feces; including, but not limited to: carbohydrate content, fat content, fiber content, protein content and/or mineral content. In another aspect of the invention, a toilet may be used to detect the presence and/or concentration of one or more enzymes in urine and/or feces; including, but not limited to: amylase, carboxypeptidase, cholecystokinin, chymotrypsin, elastase, gastric inhibitory peptide, leukocyte esterase, lipase, phospholipase, secretin, somatostatin, sterol esterase and/or trypsin. The apparatus may also be used to detect the presence and/or concentration of one or more proteins in urine and/or feces.

In one aspect of the invention, test data may be combined with data uploaded by other users to examine acute population ranges and a user's relative state within the actual population range. Test data may also be evaluated longitudinally to evaluate user's relative state within population trends. In addition to unitary variable analysis, data may be examined for interactive (multivariate), exponential, logarithmic and other effects. Combined data may be continuously evaluated for predictive or excludability potential.

In one aspect of the invention, applications that collect non-diagnostic data that may be relevant to health may be integrated into the system's data.

In another aspect of the invention, non-test data may be folded into the models both for predictive relevance and sometimes as the key measurable.

In one aspect of the invention, users may be able to set personal preferences for a variety of features; including, but not limited to: communications and alerts, test sensitivity and/or potential out-of-range conditions, PINs, information sharing, and/or specific health aspects they would like targeted for evaluation. Users may also be able to enter personal information into the system; including, but not limited to: name(s) of healthcare provider(s), health information and insurance information. Users may also be able to determine who may receive what information.

In one aspect of the invention, out of range conditions, low-probability changes to baseline metrics, trend changes or other predictive results may generate an alert. The alerts may be conveyed to a user based upon their preferences and may also be conveyed to others along with appropriate information based upon the user preferences.

In one aspect of the invention, health practitioners may have the ability to register with a system and become connected to their patient's health information, provided the patient authorizes such a disclosure. Practitioners may add their diagnoses and prescribed treatments to the system and see the impacts to patient health outcomes real-time. These diagnoses and prescriptions will be added to the overall master database to assist in uncovering new trends and correlations.

Although the present invention has been described and illustrated in detail, it is clearly understood that the same is by way of illustration and example only and is not to be taken by way of limitation, the spirit and scope of the present invention being limited only by the terms of the appended claims.

What is claimed is:

1. A human health property monitoring system, comprising:
a human waste receptacle for collecting human waste from a user;
one or more analytical instruments connected to the human waste receptacle and configured to analyze one or more property of the human waste collected by the human waste receptacle;
an electronic storage medium configured to store longitudinal data corresponding to the one or more properties of the human waste, wherein the longitudinal data comprises a statistically significant plurality of data sets corresponding to individual waste samples collected over a period of time sufficient to establish a statistically significant baseline or trend of the one or more properties; and
a computer processor configured to determine a statistically significant attribute of the longitudinal data.

2. The human health property monitoring system of claim 1, wherein the longitudinal data corresponding to the one or more property of the human waste further comprises a time component selected from one or more of a date, a time and a frequency related to when the human waste was collected.

3. The human health property monitoring system of claim 1, further comprising an input to receive a user identification corresponding to a source of the human waste.

4. The human health property monitoring system of claim 3, wherein the user identification corresponds to a single individual.

5. The human health property monitoring system of claim 3, wherein the user identification comprises demographic information corresponding to an individual.

6. The human health property monitoring system of claim 5, wherein the demographic information is anonymized.

7. The human health property monitoring system of claim 1, wherein the statistically significant attribute corresponds to data from a single individual.

8. The human health property monitoring system of claim 1, wherein the longitudinal data comprises a statistically significant plurality of data sets corresponding to individual human waste events over a period of time sufficient to establish a statistically significant deviation from a baseline, a trend, or from a population or sub-population norm.

9. The human health property monitoring system of claim 8, wherein the statistically significant deviation constitutes a statistically significant pre-symptomatic deviation.

10. The human health property monitoring system of claim 8, wherein the computer processor further comprises a notice routine configured to send an electronic notice of the statistically significant deviation to a designated recipient.

11. The human health property monitoring system of claim 8, wherein the system further comprises a diagnostic routine configured to send an electronic diagnosis of the statistically significant deviation to a designated recipient.

12. The human health property monitoring system of claim 1, wherein the analytical instrument comprises an optical, magnetic or resonant spectrometer.

13. The human health property monitoring system of claim 1, wherein the computer processor and electronic storage medium is configured to communicate with a plurality of human waste receptacles.

14. The human health property monitoring system of claim 13, wherein the plurality of human waste receptacles each comprises an input to receive a user identification corresponding to an individual user who is the source of the human waste, and wherein the data comprises the user identification corresponding to the individual user.

15. The human health property monitoring system of claim 14, wherein the plurality of human waste receptacles each comprises an input to receive a user identification corresponding to an individual user who is the source of the human waste, wherein the data comprises a plurality of user identifications corresponding to a plurality of users.

16. The human health property monitoring system of claim 15, wherein the plurality of users comprises a sufficient number of users to establish a statistically significant population or sub-population norm.

17. The human health property monitoring system of claim 16, wherein the data is derived from a sufficient number of users to determine a statistically significant deviation from the population or sub-population norm.

18. The human health property monitoring system of claim 17, wherein the computer processor further comprises a notice routine configured to send an electronic notice of the statistically significant deviation to a designated recipient.

19. The human health property monitoring system of claim 16, wherein the data is derived from a sufficient number of users to establish a statistically significant norm of a discrete sub-population group.

20. The human health property monitoring system of claim 19, wherein the discrete sub-population group is defined by one or more of the following user characteristic or group association: a medical practice group, hospital, school, prison, business group, age, blood glucose, blood pressure, body mass index, current medications, past medications, diagnoses, dietary patterns, elevation, gender, general geographic location, height, independent laboratory results, medical diagnostic test results, medical history, race, temperature, wearable device results or weight.
